(19) United States
(12) Patent Application Publication
(10) Pub. No.: US 2025/0099726 A1
(43) Pub. Date: Mar. 27, 2025

(54) AUTOMATIC CONTROL OF A THERAPY EFFICIENCY OF A SHUNT SURGERY (OF PATIENTS WITH LIVER CIRRHOSIS)

(71) Applicant: BIOTRONIK SE & Co, KG, Berlin (DE)

(72) Inventors: Franziska WEGERICH, Berlin (DE); Christian MOSS, Falkensee (DE)

(73) Assignee: BIOTRONIK SE & Co, KG, Berlin (DE)

(21) Appl. No.: 18/723,682

(22) PCT Filed: Jan. 9, 2023
(86) PCT No.: PCT/EP2023/050281

§ 371 Date: Jun. 24, 2024

(30) Foreign Application Priority Data
Jan. 10, 2022 (EP) 22150659.5

(51) Int. Cl. A61M 27/00

(52) U.S. Cl. CPC A61M 27/002; A61M 2205/3344; A61M 2210/1071; A61M 2210/12

(57) ABSTRACT

A stent system comprising a stent, a first pressure sensor and a second pressure sensor, wherein the first pressure sensor and the second pressure sensor are configured for communication with each other.

### CROSS-REFERENCE TO RELATED APPLICATIONS

This application is the United States National Phase under 35 U.S.C. § 371 of PCT International Patent Application No. PCT/EP2023/050281, filed on Jan. 9, 2023, which claims the benefit of European Patent Application No. 22150659.5, filed on Jan. 10, 2022, the disclosures of which are hereby incorporated by reference herein in their entireties.

### TECHNICAL FIELD

The present disclosure generally relates to a stent system having several pressure sensors in communication with each other and corresponding methods.

### BACKGROUND

Patients suffering from liver cirrhosis oftentimes require a shunt surgery to decrease the pressure in the liver's portal vein to reduce the risk for harmful complications such as, e.g., a lethal haemorrhage.

However, such a shunt surgery may nevertheless require regular check-ups to ensure the long-term success of the shunt implantation. It may therefore be foreseen that a patient with a shunt implant is required to see a doctor on a regular basis. A doctor may survey the therapy success of the shunt surgery, e.g., by means of a sonography and/or a catheter check-up, to exclude a shunt stenosis or a stent occlusion which may occur in approx. 50% of all surgery cases. This common procedure suffers from the disadvantage that a patient may only be able to verify the therapy success with the help of a doctor and only by being physically present in a doctor's office. Moreover, said check-ups may only occur every couple of weeks/months.

In some cases, it may be required that the check-up is based on an invasive catheter check-up which may be seen as an additional risk for the health state of the patient, in particular, if the invasive check-up is required multiple times.

Therefore, there is a need to improve the surveillance of the therapy efficiency associated with a shunt implantation.

The present disclosure is directed toward overcoming one or more of the above-mentioned problems, though not necessarily limited to embodiments that do.

### SUMMARY

This need is at least in part met by a first aspect of the present application which relates to a stent system. The stent system may comprise a stent, a first pressure sensor and a second pressure sensor. The first pressure sensor and the second pressure sensor may be configured for (wireless) communication with each other.

This implantable stent system may provide the beneficial effect that an autonomous and continuous (e.g., without being limited to check-ups at a doctor which may only occur every couple of weeks/months) monitoring of a pressure difference between two sensors of the stent system is provided in a miniaturized system associated with an implanted shunt. Hence, the pressure drop across the stent may be determined such that an occlusion or stenosis may be detected early on. In particular, it may be foreseen that at least one of the first pressure sensor and/or the second pressure sensor may initiate a pressure measurement autonomously (e.g., without an external activation such as by the patient). This may allow for a regular (e.g., every minute, every hour, every day, every week, etc.) monitoring of a therapy efficiency without the requirement of regularly attending medical check-ups. Therefore, the disadvantage of a check-up which may only occur every couple of weeks/months and which may only show a snapshot of the current therapy success may be successfully overcome. A potential stenosis or occlusion may thus be discovered at an early stage and harmful effects for the health state of the patient may be avoided, e.g., by applying a respective counter-therapy (e.g., by means of administering respective drugs such as, e.g., a certain amount of diuretics).

By means of the communication, one of the sensors may inform the other about a measurement time, such that both may make measurements at the same time. The communication may be unidirectional. In other examples, it may be bidirectional (in which case the “other” sensor may also transmit the measurement data to the “one” of the sensors).

It has further been observed that regular check-ups at a doctor's office may suffer from the compliance of the patient, i.e., the aspect how often the patient follows the recommendation of a doctor to attend regular check-ups and whether the patients attend check-ups regularly post-surgery. It has been shown that the patient compliance may decrease over time, i.e., a patient may tend to less strictly follow recommendations to attend regular check-ups the longer the time distance between the surgery and the potential check-ups. Also, this drawback may be overcome by the present disclosure, as a constant evaluation whether the therapy success of the patient has deteriorated may be facilitated.

It may also be foreseen that the stent system is equipped with one or more further pressure sensors (e.g., at least with a third pressure sensor). The further pressure sensors may be in (wireless) communication with the first pressure sensor and/or the second pressure sensor and/or with each other.

The first pressure sensor and the second pressure sensor may be fixed to the stent.

The first pressure sensor and the second pressure sensor may be arranged at different positions along a longitudinal axis of the stent. The stent may possess an inlet (through which blood may enter the stent) and the stent may possess an outlet (through which blood is exiting the stent). For example, the stent may comprise a total length (e.g., from the inlet to the outlet), along which a longitudinal axis may extend. It may be possible that the first pressure sensor is closer to the inlet than it is to the outlet, for example it may be approximately at the inlet (or vice versa). The second pressure sensor may be closer to the outlet than it is to the inlet, for example it may be approximately at the outlet (or vice versa). It may also be possible that the first pressure sensor and the second pressure sensor are symmetrically arranged about at a center portion of the stent (e.g., the distance from the center portion to the inlet may be equal to the distance from the center portion to the outlet). In such a case the first pressure sensor and the second pressure sensor may be placed at a certain (equal) distance from the center of the stent. Alternatively, any other distribution of the first pressure sensor and the second pressure may also be possible. However, it may be preferred that the first pressure sensor and the second pressure sensor are at least a certain distance apart from each other along the longitudinal axis of the stent (e.g., at least 1 cm, 2 cm, 3 cm, 4 cm, or 5 cm, etc.).

This arrangement of the first pressure sensor and the second pressure sensor may allow for a measurement of an artery pressure at two (different) locations and may allow for a determination of a location at which a stenosis and/or an occlusion has occurred.

The stent may be configured to be implanted during a transjugular intrahepatic portosystemic shunt (TIPS) surgery, e.g., into a liver vein. The stent may preferably be implanted such that it connects a portal artery of the liver with a liver vein, preferably a right liver vein.

By implanting the stent as described beforehand, it may be ensured that a potential increase of a blood pressure (which may occur due to a stenosis or a occlusion) may be detected, such that harm to the liver and/or further organs before the liver may be avoided.

At least one of the pressure sensors may be an active pressure sensor. The first pressure sensor and/or the second pressure sensor may be equipped with a (rechargeable) battery. The first pressure sensor and/or the second pressure sensor may not depend on an external excitation field for supplying the first pressure sensor and/or the second pressure sensor with power (e.g., by means of electromagnetic induction).

This implementation may provide the advantage that a patient is not required to initiate an acquisition of data, e.g., by means of placing induction and readout electronics on his/her chest. Therefore, a regular surveillance of the therapy success associated with the implantation of the stent may (automatically) be facilitated.

It is noted that in some examples a stent system may be provided that comprises a stent, and a first pressure sensor and a second pressure sensor, wherein at least one of the pressure sensors is an active pressure sensor. In such examples, the pressure sensors do not necessarily need to be configured for communication with each other.

The communication may be based at least in part on a capacitive coupling between the first pressure sensor and the second pressure sensor.

The stent may generally be made from an electrically conductive material and/or may be coated with an electrically conducting material. By means of the capacitive coupling it may be foreseen that the first pressure sensor is conductively connected to the second pressure sensor by means of the stent. In such an implementation, the stent essentially acts as an electric wire.

It may further be foreseen that at least a portion of the first pressure sensor and/or the second pressure sensor is electrically isolated (it may still be electrically conducting) from the remaining part of the first pressure sensor and/or the second pressure sensor, respectively.

The remaining parts of the first pressure sensor and/or the second pressure sensor may be electrically connected to the stent. If the first pressure sensor and the second pressure sensor comprise an electrically isolated portion, the isolated portions may be in capacitive coupling with each other, which may be used for wireless communication between the first and second pressure sensors.

By means of the capacitive coupling of the first pressure sensor and the second pressure sensor, it may be facilitated that a wireless sensor-to-sensor communication is enabled without the requirement for electromagnetic transmission electronics such as, e.g., antennas and associated amplification circuits. This may decrease the system complexity and may decrease the required power consumption.

The first pressure sensor may be configured to activate the second pressure sensor. For example, it may be foreseen that the second pressure sensor is in a sleep mode as a default mode. The second pressure sensor may thus be switched off for most of the time and may only be activated (e.g., such that the second pressure sensor leaves the sleep mode), if a wake-up signal is received from the first pressure sensor. The wake-up signal may preferably be received by means of a capacitive coupling between the first pressure sensor and the second pressure sensor. The second pressure sensor may be configured to acquire pressure data after having received the wake-up signal. The first pressure sensor may be configured to acquire pressure data after having sent the wake-up signal such that its acquisition is synchronous with that of the second pressure sensor (e.g., taking into account signal processing and wake-up time of the second pressure sensor).

Additionally or alternatively, it may also be foreseen that the second pressure sensor is activated by an external device, e.g., a patient device (e.g., a smartphone, an OEM device, a wearable (e.g., a smartwatch, etc.).

By implementing the second pressure sensor such that it is activated by the first pressure sensor, the overall energy consumption of the stent system may be minimized as energy may only be consumed if the first pressure sensor wakes up the second pressure sensor for, e.g., a regular acquisition of pressure data.

The second pressure sensor may be configured to transmit acquired pressure data to the first pressure sensor. For example, if the second pressure sensor is activated, the second pressure sensor may acquire pressure data (which may be associated with the blood pressure at the location at which the second pressure sensor is located). The second pressure sensor may locally store the acquired pressure data and/or may transmit the acquired pressure data to the first pressure sensor.

By transmitting the pressure data to the first pressure sensor, the second pressure sensor may possess a minimalistic hardware design. In particular, the second pressure sensor may not require complex processing electronics (such as, e.g., a processor or any other central processing unit (CPU)) and may thus only require a low capacity battery. This may decrease the overall complexity, size and cost of the stent system and may decrease the overall energy consumption. In such a case, still the first pressure sensor may be equipped with (more energy intensive) processing electronics.

At least one of the pressure sensors may comprise a waveform generator. A waveform generator may relate to a generator which is capable of providing a certain waveform signal (e.g., a voltage vs. time signal). The waveform generator may be configured to provide a single or it may be configured to provide one or more different types of waveform signals. The provided signal may, e.g., relate to a sinusoidal signal, a ramp signal, a sawtooth signal and/or any other signal.

The waveform generator may be configured to vary (according to the provided waveform) the charge on the portion of the first pressure sensor or the portion of the second pressure sensor, respectively, which is electrically isolated from the remaining portion of the respective sensor as it has further been described above. As a consequence of the conductive coupling of the first pressure sensor and the second pressure sensor, the charge on the respective other pressure sensor (which may not necessarily be modulated with a waveform generator) may also be varied. This may be understood as the wireless communication between the first pressure sensor and the second pressure sensor as it has been described above.

The stent system according to any of the preceding claims, may further comprising means for determining a pressure gradient across the stent based at least in part on pressure data acquired by the first pressure sensor and pressure data acquired by the second pressure sensor. The pressure gradient may relate to a pressure difference derived from the acquired pressure signal acquired by the first pressure sensor and the acquired pressure signal acquired by the second pressure signal. The pressure difference may optionally further be normalized to the distance between the first pressure sensor and the second pressure sensor. This may allow the determination of a potential pressure drop per unit length in between the first pressure sensor and the second pressure sensor. The determination of the pressure gradient may allow a reliable conclusion whether the patient is at risk for a stenosis and/or an occlusion.

The stent system may further comprise an interface for communicating with at least one other implant and/or external device. The interface for communicating may relate to hardware (e.g., antennas, amplifiers, filters) and/or software (e.g., protocols) means for communication. The means may preferably allow for a wireless communication with the at least one other implant and/or the external device. The communication may relate to receiving programming commands (at the stent system) from the at least one other implant and/or the external device. The programming commands may, e.g., relate to configurations which may, e.g., define the time interval at which pressure data may be acquired by the first pressure sensor and/or the second pressure sensor. The programming data may also relate to a request associated with status information of the first pressure sensor and/or the second pressure sensor (e.g., directed to a current battery level, a current firmware version, etc.).

The interface may further be adapted to allow, e.g., for intrabody communication (IBC) if the communication is performed with at least one other implant. If the communication is performed with an external device, the interface may allow for MICS-telemetry, Wi-Fi, Bluetooth (Low Energy), 5G, etc. In any case, the communication may be encrypted.

For example, the interface may be comprised by the first pressure sensor. Commands as to the measurement times may be received. At the respective measurement times, the first pressure sensor may wake up the second pressure sensor and both may carry out pressure measurements at the same time.

The interface may in particular provide the advantageous effect that the configuration of the stent system may be adapted to varying characteristics of the patient (e.g., a potential deterioration of the health state of the patient) after implantation.

The interface may be configured to transmit pressure data acquired by the first pressure sensor and/or the second pressure sensor to the at least one other implant and/or external device.

The interface, as described beforehand, may further be adapted such that acquired pressure data may be transmitted to at least one other implant for further processing (e.g., for calculating a pressure gradient, for storing the acquired pressure data, etc.). The at least one other implant may also act as a relay device which may only forward pressure data (e.g., to an external device) transmitted to the at least one other implant without further processing of the data. It may be foreseen that the interface transmits the individually acquired pressure data to at least one other implant and/or external device as described beforehand.

Additionally or alternatively, it may also be foreseen that the interface transmits a data associated with a pressure gradient/drop to the at least one other device and/or the external device.

The external device may be implemented as described above. Moreover, the external device may also be implemented as a dedicated readout device for the stent system which may, e.g., be administrated by the manufacturer of the stent system. The dedicated readout device may be operated by a doctor.

The transmission of any acquired pressure data to at least one other implant and/or the external device may allow a further monitoring of the pressure data by a patient and/or doctor and a tracking of a potential evolution of the pressure data.

Another aspect of the present application relates to a method for monitoring an intravascular pressure. The method may comprise the step of acquiring pressure data by a first pressure sensor and a second pressure sensor of a stent system. The first and the second pressure sensor may be in (wireless) communication with each other.

The first pressure sensor and the second pressure sensor may be implemented as described above. The communication between the first pressure sensor and the second pressure sensor may allow for a simultaneous acquisition (e.g., synchronized to occur at the same time) of pressure data. In some examples, the step of acquiring pressure data relates to acquiring pressure data synchronously be the first and second pressure sensors, e.g., based on the (wireless) communication with each other. This may in particular allow for a correlation of the acquired pressure data acquired by the first pressure sensor and of the acquired pressure data acquired by the second pressure sensor (preferably at different locations). It is noted that the acquisition of pressure data by the first pressure sensor and/or the second pressure sensor may also be done disentangled from each other, e.g., not (time) synchronized. In such a case, only one of the pressure sensors may acquire pressure data or one of the pressure sensors acquires pressure data whereas the respective other pressure sensor acquires pressure data after a certain pre-defined time interval (e.g., 1 s, 1 min, 10 min, 1 h, etc.).

The method may further comprise activating, by the first pressure sensor, the second pressure sensor. In such a case, the second pressure sensor may only acquire pressure data upon activation by the first pressure sensor to conserve energy.

The method may further comprise receiving, by the first pressure sensor, pressure data acquired by the second pressure sensor. In such an implementation, a (centralized) data processing comprising the first pressure data from the first pressure sensor and pressure data from the second pressure sensor may be facilitated, e.g., as it has further been described above.

The method may further comprise determining a pressure difference based at least in part on the pressure data acquired by the first pressure sensor and the pressure data acquired by the second pressure sensor. The pressure difference may additionally be normalized to the distance between the first pressure sensor and the second pressure sensor such as to obtain a pressure gradient (e.g., a pressure change/drop per unit distance). The normalization may, e.g., allow for easier comparability of the pressure change (e.g., over a tracked time evolution and/or among several patients who may have different stent systems (e.g., comprising different distances between the first pressure sensor and the second pressure sensor)). In any case, this may allow for an increased reliability when determining whether a patient suffers from a stenosis and/or an occlusion. The determining may be performed by the stent system itself, e.g., the first pressure sensor, or it may be performed by another implant and/or external device to which the first and second pressure data has been transmitted.

It is noted that the term “implantable” refers to elements that may not have been but are configured for being implanted into a patient. However, the term “implantable” also includes elements that have already been implanted.

The functions/method steps described herein may generally be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the present disclosure. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations.

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media may comprise RAM, ROM, electrically erasable programmable read only memory (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Combinations of the above are also included within the scope of computer-readable media.

It is noted that all aspects described may be implemented as method steps, even if described with reference to a device or system. On the other hand, it is understood that the devices and systems described herein may be equipped with means implementing functionalities that may only be described herein with reference to method steps.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The following figures are provided to support the understanding of the present invention:

FIG. 1 Illustration of an exemplary implementation of a stent system after a TIPS surgery,

FIG. 2 Schematic illustration of an exemplary stent system,

FIG. 3 Schematic illustration of a stent system and a capacitive coupling of a first pressure sensor and a second pressure sensor of the stent system,

FIG. 4 Illustration of an exemplary equivalent circuit diagram of the capacitive coupling of a first pressure sensor and a second pressure sensor,

FIG. 5 Alternative illustration of an exemplary equivalent circuit diagram of the capacitive coupling of a first pressure sensor and a second pressure sensor.

### DETAILED DESCRIPTION

The following detailed description outlines possible exemplary embodiments of the present invention.

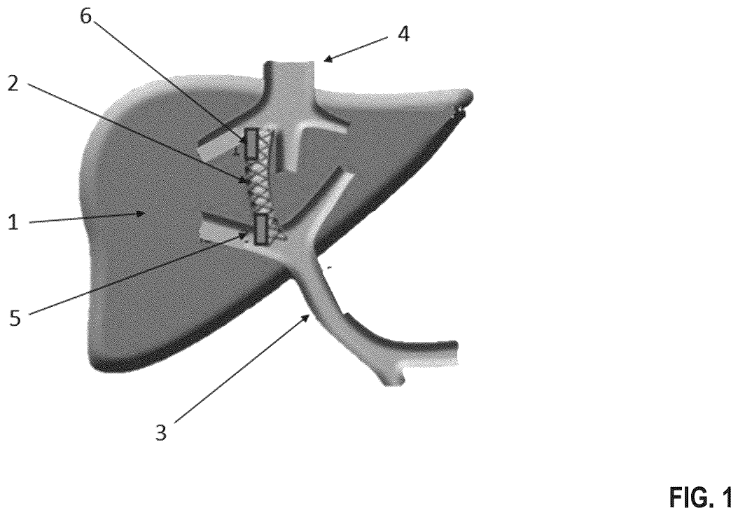

FIG. 1 shows an exemplary implementation of a stent system after a TIPS surgery. Liver 1 which may be equipped with the stent system. Liver 1 may be a human liver.

The stent system may be implemented such that a blood vessel (not shown in FIG. 1) in between a portal artery 3 and a right liver vein 4 may additionally be supported such that any blockage of the blood vessel (which may, e.g., arise from a pre-existing disease such as, e.g., liver cirrhosis) is avoided by means of stent 2. In other words, by means of the implantable stent system, the respective blood vessel may additionally experience structural support such that a total blockage or at least a partial blockage of the blood vessel may be avoided. However, it may also be required to perform a surveillance of the success of said therapy (i.e., the implantation of the stent system system) which may be negatively affected, if a blockage of the stent 2 occurs. Namely, a blockage of said blood vessel (or the stent 2) may lead to an increase of the blood pressure in the portal artery 3 and in organs which were perfused with blood prior to entering the portal artery 3.

Right liver vein 4 may return blood, which has flown through liver 1, to the heart (not shown in FIG. 1) of the patient. It is acknowledged that right liver vein 4 is only shown exemplarily and that it may also be possible to implant the stent system (and in particular stent 2) such that it may connect to any vein in the left hepatic lobe (not shown in FIG. 1).

Stent 2 may be equipped with a first pressure sensor 5 and a second pressure sensor 6. The first pressure sensor 5 and the second pressure sensor 6 may be implemented as it is described herein. They may be used to measure a pressure gradient/drop from the portal artery 3 to, e.g., the right liver vein 4 (e.g., along the length axis of the stent).

It may in particular be possible that the first pressure sensor 5 and the second pressure sensor 6 are in wireless communication with each other. The wireless communication may be implemented as any wireless communication technology (such as, e.g., MICS-telemetry, Wi-Fi, Bluetooth (Low Energy), near field communication (NFC), intrabody communication (IBC), LTE, 5G, etc.). The wireless communication may additionally or alternatively be based at least in part on ultrasound and/or a galvanic communication. Additionally or alternatively, it may also be possible that the wireless communication is based at least in part on a capacitive coupling of the first pressure sensor 5 and the second pressure sensor 6.

Any of the above-mentioned wireless communication technologies may also be used to communicate with at least one other implant and/or an external device, as described herein (not shown in FIG. 1). Any of the above-mentioned communication technologies may be implemented unidirectionally and/or bidirectionally.

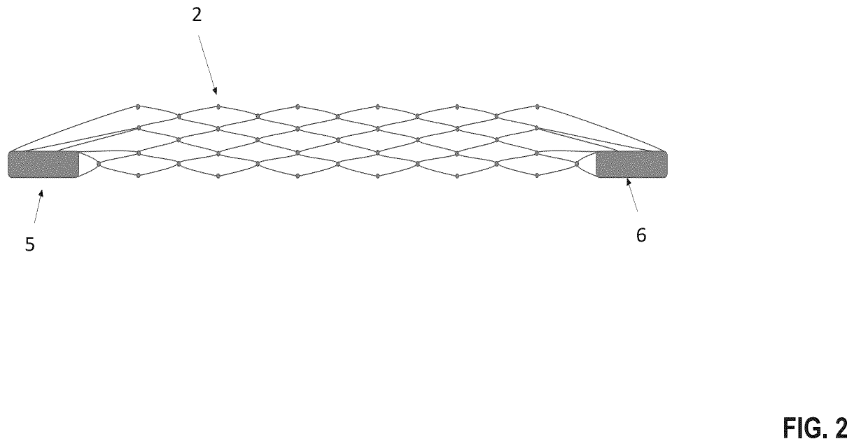

FIG. 2 exemplarily shows a side view of the exemplary stent system. The stent system may comprise a stent 2. Stent 2 may comprise an inlet (e.g., right side in FIG. 2) and an outlet (e.g., left side in FIG. 2) between which a longitudinal axis of the stent 2 extends. The stent 2 may have an approximately circular cross-section. Stent 2 may have a tubular shape (however, in other examples also other shapes may be used). A tubular shape may refer to a shape wherein the length of the stent (along the longitudinal axis) is at least twice the radius of the stent, for example.

The stent 2 may be made from an electrically conducting material (and/or may be coated with an electrically conducting material) and may be manufactured in a grid-like pattern.

In a preferred embodiment, stent 2 may be terminated by the first pressure sensor 5 and the second pressure sensor 6 which may preferably be arranged at a maximum distance apart from each other to acquire pressure data (associated with a blood pressure at the respective locations of the first pressure sensor 5 and the second pressure sensor 6). However, it may also be possible that the first pressure sensor 5 and the second pressure sensor 6 are located at any other location along (the longitudinal axis of) the stent 2. Apart from that, it may be possible to orient the first pressure sensor 5 and/or the second pressure sensor 6 at any rotational location around the cross-section of the stent 2.

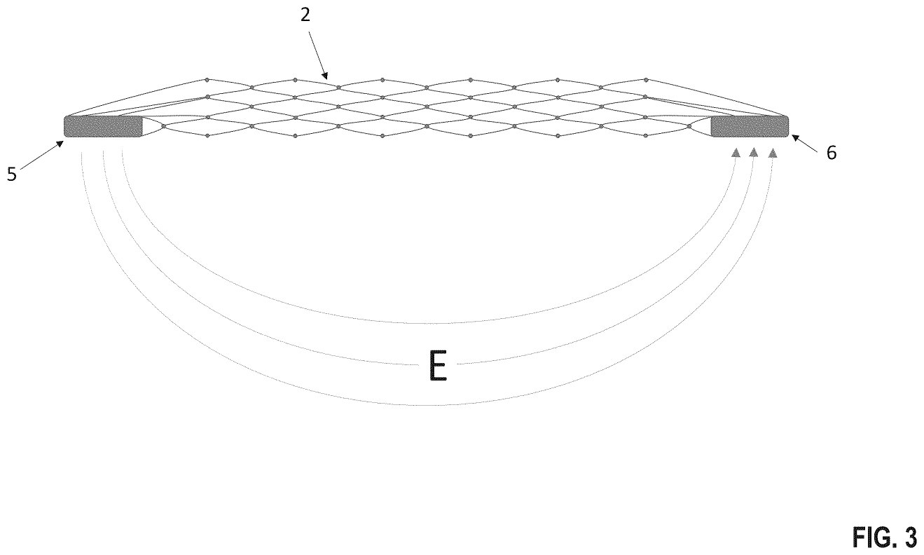

FIG. 3 shows a schematic illustration of the stent system comprising stent 2, first pressure sensor 5 and second pressure sensor 6 and illustrates the capacitive coupling between the first and second pressure sensors 5/6.

It may be foreseen that the first pressure sensor 5 and the second pressure sensor 6 are electrically connected to the stent 2, wherein the stent 2 may be electrically conducting. It may further be foreseen that an electric field E is generated between the first pressure sensor and the second pressure sensor 6 such that the electric field lines may preferably originate from the first pressure sensor 5 and may evolve such that the electric field lines terminate at the second pressure sensor 6. Since the electric field lines associated with the electric field E arise from a positive electric charge at the first pressure sensor 5 and a negative electric charge at the second pressure sensor 6, the electric field E may lead to an equalization of the associated electric charge imbalance by causing an electric current flow, e.g., from the second pressure sensor 6 to the first pressure sensor 5. The current flow may, e.g., occur by means of the stent 2.

It is acknowledged, that the electric field E may also arise from the second pressure sensor 6 and may terminate at the first pressure sensor 5 (thus vice versa to the exemplary embodiment as outlined beforehand). In such an implementation a current flow may occur from the first pressure sensor 5 to the second pressure sensor 6.

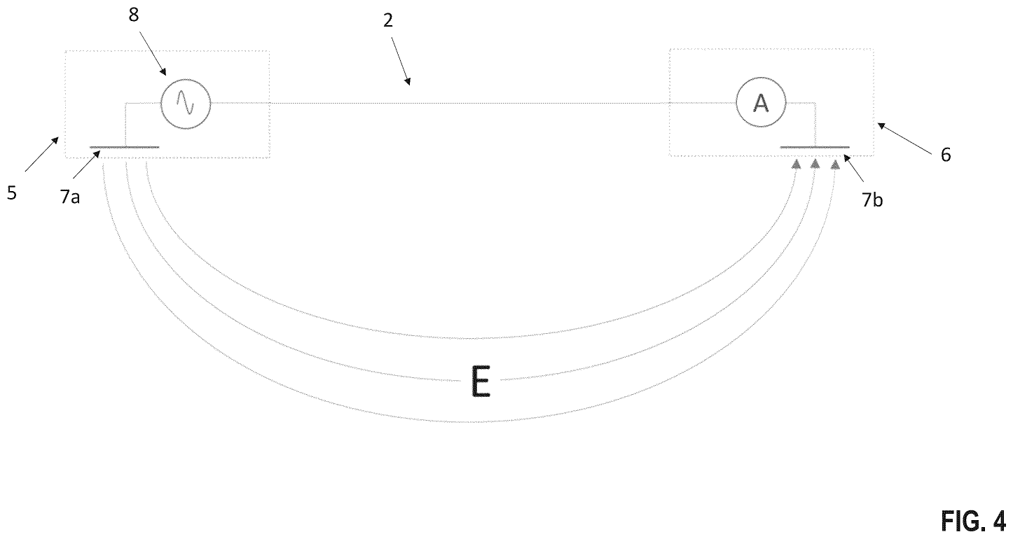

FIG. 4 shows an illustration of an exemplary equivalent circuit diagram (e.g., of FIG. 3) of the capacitive coupling between a first pressure sensor 5 and a second pressure sensor 6. The first pressure sensor 5 and/or the second pressure sensor 6 and the stent 2 may be implemented as it has been described above (e.g., with reference to FIGS. 1-3).

Pressure sensor 5 may be electrically connected to the second pressure sensor 6 by means of its housing and stent 2 (only shown schematically in FIG. 4), e.g., by means of a dedicated conducting surface portion of the housing of pressure sensor 5 and, optionally a corresponding portion of the housing of pressure sensor 6.

Additionally, each of pressure sensor 5 and pressure sensor 6 may comprise a conducting surface 7a, 7b which is electrically isolated from the connection between first and second pressure sensors 5/6. Conducting surfaces 7a, 7b may be implemented equally.

Additionally, the first pressure sensor 5 may comprise a voltage source, e.g., a waveform generator 8. Waveform generator 8 may be configured to generate one or more different modulation signals (as outlined herein) which may be used to change the amount of charge on conducting surface 7a (e.g., of the first pressure sensor 5) vs. time (e.g., the charge on conducting surface 7a may be different for different points in time).

Waveform generator 8 may provide a carrier frequency upon which payload data may be imprinted (e.g., by means of an amplitude modulation (AM) and/or a frequency modulation (FM) and/or a pulse width modulation (PWM) and/or any other suitable technique).

By means of the electric field E arising from the conducting surface 7a of the first pressure sensor 5, terminating at the conducting surface 7b of the second pressure sensor 6, an electric charge may be induced on the conducting surface 7b of the second pressure 6 which may be equal to the charge on the conducting surface 7a of the first pressure sensor 5 but may possess an inverted sign (e.g., if the conducting surface 7 of the first pressure sensor 5 is positively charged, the conducting surface 7 of the second pressure sensor 6 may be negatively charged or vice versa). The charge on the conducting surface 7b of the second pressure sensor 6 may, e.g., be supplied by means of the electrically conducting connection between the first pressure sensor 5 and the second pressure sensor 6 by means of the stent 2. Therefore, any change of the charge on the conducting surface 7a (e.g., by means of the waveform generator 8) of the first pressure sensor 5 may automatically cause a change of the charge on the conducting surface 7b of the second pressure sensor 6 due to the capacitive coupling.

Therefore, the outlined mechanism may provide the wireless communication capability as disclosed by the present application.

As outlined above, it may be foreseen that the second pressure sensor 6 is in a sleeping mode per default to minimize energy consumption. It may be possible that the second pressure sensor 6 may receive a wake-up command from the first pressure sensor 5 via capacitive coupling, as it will further be described below.

Furthermore, the second pressure sensor 6 may comprise a (signal) processing unit A. Processing unit A may comprise an ampere meter, amplifier, a filter, a threshold detector (e.g., trigger), an ADC, a DAC, a demodulator etc. In some embodiments, it may also be foreseen that also the first pressure sensor 5 comprises a signal processing unit A.

The current flow as described above, may, e.g., be sensed by the signal processing unit A of the second pressure sensor 6. If said current flow exceeds a certain threshold, signal processing unit A may interpret the exceedance as a command, received from the first pressure sensor 5, that the second pressure sensor 6 should leave the sleeping mode and that it should start active operation, e.g., carry out a pressure measurement.

In some embodiments, the second pressure sensor 6 may not comprise a waveform generator 8, such that the communication may be unidirectional (the second sensor may, in such as case, have a transmitter to transmit pressure data to another implantable or external device).

However, additionally or alternatively, it may also be possible that the second pressure sensor 6 comprises a waveform generator 8 which may be implemented identically to the waveform generator of the first pressure sensor 5, for example. Then, the current flow between the second pressure sensor 6 and the first pressure sensor 5 may be used to transmit pressure data, e.g., from the second pressure sensor 6 and the first pressure sensor 5.

In an exemplary embodiment, it may be foreseen that a timer (which may be comprised by the first pressure sensor 5 or which is in communication with the first pressure sensor) initiates the first pressure sensor 5 to acquire pressure data.

Upon triggering the acquisition by means of the above-mentioned timer, the first pressure sensor 5 may first activate the second pressure sensor 6 (e.g., by means of the capacitive coupling) such that also the second pressure sensor 6 is triggered to acquire respective pressure data. Both pressure sensors 5 and 6 may then acquire the pressure data. The acquisition of pressure data by the first pressure sensor 5 and the second pressure sensor 6 may thus be synchronous. Optionally, the second pressure sensor 6 may transmit acquired pressure data to the first pressure sensor 5 by means of the capacitive communication link.

In that case, it may be foreseen that also the second pressure sensor 6 comprises a waveform generator 8.

In an alternative embodiment, it may also be possible that the timer triggers the second pressure sensor 6 to acquire pressure data. In such an implementation, the second pressure sensor 6 may activate the first pressure sensor 5 to acquire pressure data which may then be transmitted to the second pressure sensor 6.

Additionally or alternatively, it may also be foreseen that the first pressure sensor 5 and the second pressure sensor 6 are both triggered externally, e.g., by means of an interface (as described above). In such an implementation, a trigger for the acquisition of pressure data may be received from at least one other implant and/or an external device. In the latter case, the trigger may be initiated by the patient, a doctor, a relative, etc. The triggering may occur such that a simultaneous acquisition of pressure data by the first pressure sensor 5 and the second pressure sensor 6 is initialized. However, it may also be foreseen that the acquisition of pressure data by the second pressure sensor 6 occurs a certain pre-defined time interval after the acquisition of pressure data by the first pressure sensor 5 (or vice vice). In may further be foreseen that the capacitive coupling between the first pressure sensor 5 and the second pressure sensor 6 is then used to transmit pressure data from the second pressure sensor 6 to the first pressure sensor 5 (or vice versa).

It is further acknowledged that all features described above for the first pressure sensor 5 may also be implemented in the second pressure sensor 6 (and vice versa).

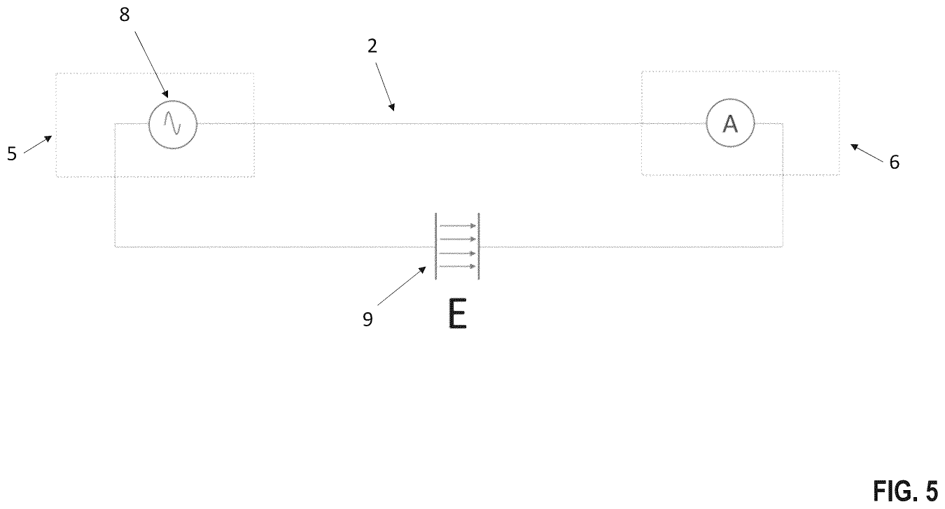

FIG. 5 shows a further exemplary equivalent circuit diagram following FIG. 4. FIG. 5 shows the stent system according to the present application, comprising stent 2 and the first pressure sensor 5 and the second pressure sensor 6. Stent 2, the first pressure sensor 5 and the second pressure sensor 6 may be implemented as it has been described above with reference to any of FIGS. 1-4. FIG. 5 shows the capacitive coupling between the first pressure sensor 5 and the second pressure sensor 6 in a different manner than FIG. 4, namely, by equivalent parallel capacitor plates 9 that would form a capacitor having a similar capacitance as provided between conducting surfaces 7a and 7b of FIG. 4.

Plates 9 may be understood to be separated from each other by a certain distance d. In other words, conducting surfaces 7a, 7b may essentially define a capacitor with parallel capacitor plates 9, wherein the capacitance may depend on a surface area of the conducting surfaces 7a, 7b and the distance of the with conducting surfaces 7a, 7b.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A stent system, comprising:
a stent;
a first pressure sensor and a second pressure sensor;
wherein the first pressure sensor and the second pressure sensor are configured for communication with each other.

2. The stent system of claim 1, wherein the first pressure sensor and the second pressure sensor are arranged at different positions along a longitudinal axis of the stent.

3. The stent system of claim 1, wherein the stent (2) is configured to be implanted into a liver vein, in particular during a transjugular intrahepatic portosystemic shunt (TIPS) surgery.

4. The stent system of claim 1, wherein at least one of the pressure sensors is an active pressure sensor.

5. The stent system of claim 1, wherein the communication is based at least in part on a capacitive coupling between the first pressure sensor and the second pressure sensor.

6. The stent system according to claim 1, wherein the first pressure sensor is configured to activate the second pressure sensor.

7. The stent system according to claim 1, wherein the second pressure sensor is configured to transmit acquired pressure data to the first pressure sensor.

8. The stent system according to claim 1, wherein at least one of the pressure sensors comprises a waveform generator.

9. The stent system according to claim 1, further comprising means for determining a pressure gradient across the stent based at least in part on pressure data acquired by the first pressure sensor and pressure data acquired by the second pressure sensor.

10. The stent system according to claim 1, wherein the stent system further comprises an interface for communicating with at least one other implant and/or external device.

11. The stent system according to claim 10, wherein the interface is configured to transmit pressure data acquired by the first pressure sensor and/or the second pressure sensor to the at least one other implant and/or external device.

12. A method for monitoring an intravascular pressure, comprising:
acquiring pressure data by a first pressure sensor and a second pressure sensor of a stent system;
wherein the first pressure sensor and the second pressure sensor are in communication with each other.

13. The method of claim 12, further comprising:
activating, by the first pressure sensor, the second pressure sensor.

14. The method of claim 12, further comprising:
receiving, by the first pressure sensor pressure data acquired by the second pressure sensor.

15. The method of claim 12, further comprising:
determining a pressure difference based at least in part on the pressure data acquired by the first pressure sensor and the pressure data acquired by the second pressure sensor.
